(19) United States
(12) Patent Application Publication
(10) Pub. No.: US 2006/0052675 A1
(43) Pub. Date: Mar. 9, 2006

(54) Method and system for the detection, amelioration and treatment of psychological trauma and other mental disorders, and for performance and creative enhancement

(76) Inventor: David Grand, Bellmore, NY (US)

Correspondence Address: Mitchell A. Stein, Esq.;STEIN LAW, P.C., Suite 4, 24 Woodbine Avenue, Northport, NY, 11768, US

(21) Appl. No.: 10/935,665

(22) Filed: Sep. 7, 2004

(51) Int. Cl. A61B 5/00

(52) U.S. Cl. 600/300

(57) ABSTRACT

A non-invasive method for permitting the processing of a triggering event, psychological or psychosomatic symptom by a recipient, involving application of tracking of at least one eye along at least two axes, determining a targeted location reflected by bodily reflex, typically an eye movement (either a jump, blink or twitch) and also including quick breaths, swallows, and facial and bodily twitches while tracking; associating the targeted location with a particular event in the recipient's life experience; fixing the recipient's gaze in the at least one eye upon the targeted location; and assisting the recipient in processing the particular event while maintaining the fixation of the eye. The system includes the addition of auditory stimuli, involving bilateral auditory stimulation, as well as a stylus for focusing the recipient's eye(s) while applying the method.

### FIELD OF THE INVENTION

The present invention relates to the field of detection, amelioration and treatment of psychological trauma and other mental disorders, and for performance and creative enhancement, and more particularly to a method and system for treating people with a combination of sensory and auditory stimulation focused on specific points in eye position to permit the person to process human experiences and traumatic events that present psychological or psychological manifestations.

### BACKGROUND OF THE INVENTION

Heretofore known in the art is a technique called eye movement desensitization and reprocessing (“EMDR”) which typically involves sweeping eye motion in a lateral mode while processing traumatic memories of earlier events in a person's life. EMDR, however, uses eyes tracking a continuously moving hand. In EMDR, the moving hand does not stop or focus on specific points, but rather continues in motion.

EMDR is well known in the art. Eye movement Desensitization and Reprocessing, Basic Principles, Protocols, and Procedures, by Francine Shapiro, 2001, the Guilford Press, 2^{nd} Edition shows fast motion of the fingers horizontally from left to right in a horizontal plane. Likewise, Toward Integration: One Eye at a Time, by Cook and Bradshaw, 2002, SightPysch Seminars Incorporated, 2^{nd} Edition, Third Printing, shows use of a one-eye technique specifically avoiding focusing on points of eye glitches or other psychologically indicators which are considered to be overstimulating to the recipient. Also, Of Two Minds, by Fredric Schiffer, 1998, the Free Press, utilizes a dual brain approach using goggles which allow light to pass into the extreme left or extreme right of the eye, without identifying or focusing upon any points in this approach. Waking the Tiger, Healing Trauma, by Peter A. Levine, 1997, North Atlantic Books, utilizes body sensation and shifting of attention in the amelioration of trauma, without use of eye movement or eye focusing.

U.S. Pat. No. 5,343,261 to Wilson shows a device for inducing saccadic eye movement of failure great complexity for applying a moving light bar for use in EMDR. U.S. Pat. No. 5,619,291 to Putnam uses two video displays and hand controllers in EMDR for displaying images that have an emotional impact, without reference to single fixed points in the tracking of either eye. U.S. Pat. No. 6,001,073 to Schmidt, et al. shows an alternating bilateral tactile stimulation and again has no reference to fixed points in eye tracking. U.S. Pat. No. 6,409,655 to Wilson, et al. shows audio stimulation, but also does not relate to focusing on fixed eye points. Lastly, U.S. Pat. No. 6,425,764 to Lamson relates a biofeedback goggle device.

The prior art has heretofore not shown, and in fact has taught away from the employment of a method and system for determining a specific, fixed location in eye movement relating to a specific traumatic or human experience.

It is therefore an object of the present invention to provide detection, amelioration and treatment of psychological trauma and other mental disorders, and for performance and creative enhancement heretofore unforseen in the art.

### SUMMARY OF THE INVENTION

The various features of novelty which characterize the invention are pointed out with particularity in the claims annexed to and forming a part of the disclosure. For a better understanding of the invention, its operating advantages, and specific objects attained by its use, reference should be had to the drawings and descriptive matter in which there are illustrated and described preferred embodiments of the invention.

The foregoing objects and other objects of the invention are achieved through a non-invasive method for permitting the processing of a triggering event by a recipient, involving application of tracking of at least one eye along at least two axes, determining a targeted location reflected by a reflexive response, like an atypical eye movement (like a jump, blink or twitch) or pupil dilation, quick inhalation and the like while tracking; associating the targeted location with a particular event in the recipient's life experience; fixing the recipient's gaze in the eye upon the targeted location; and assisting the recipient in processing the particular event while maintaining the fixation of the eye. The system includes the addition of auditory stimuli, involving bilateral auditory stimulation, as well as a stylus for focusing the recipient's eye(s) while applying the method.

An essential aspect of the subject invention is the recognition that a trigger, like a traumatic or emotional occurrence either unprocessed or improperly processed by the human mind, results in behavioral changes, like a YIPS event. A YIPS event is a physiologically reflexive response to an emotional experience. In sports, for example, a YIPS event occurs in a golfer when the putter is observed to freeze or jerk uncontrollably during a swing. Normally the swing is intended to be smooth, and a YIPS event is observed when the smoothness is interrupted.

As a person develops, triggers become buried deeply in the person's mind. In the case of the instant invention, such triggers are considered lesions (whether presenting physiologically or psychologically), and are associated with reflexive actions, including eye motion, when the person is placed under the method and system of the invention. Thus, when one or both eyes are tracked, like the swing of the putter, a spontaneous reflexive action is observed in the pattern, and fixation upon the point of reflexive action cues to the underlying trigger and experiential event, unprocessed or poorly processed by the person, resulting in imperfect human behavior.

To determine the precise location in the sweeping eye motion of the person, a stylus is employed upon which the person focuses for identification of the location, first, and then for treatment at that location. Thus, the person focuses on the stylus (typically while experiencing bilateral auditory stimulation to enhance the effect) as the stylus sweeps laterally, and once the reflexive action is observed (either a twitch, or blink of the eye or other physical manifestation like other facial actions or physical actions of limbs, etc.), that point serves as the basis for further honing. Thus, the first determination is of the lateral location of the reflexive action as indicative of a lesion in the brain. Then, the stylus is moved vertically in the plain of the lateral location to determine a greater response. Optionally, the stylus is then moved forwards or backwards to determine the location. A point is thereby determined. Observably, each lesion in the brain is evidenced by a different point for triggering reflexive physiological response. All such locations constitute a map of the brain of all such lesions.

Once a lesion is located by way of a physical location of the stylus, the person is provoked to raise the stress level to its maximum. Different from forms of EMDR which seek to sweep past such maximum stress levels, the instant invention seeks to focus upon such locations, and compel the person to process at that location.

The instant invention utilizes either each eye independently (and then together in a sweeping format as demonstrated in FIG. 2 and its description, below), or together from the outset. The stylus can take many forms, provided that a single point for focus is achieved. The method and system of the instant invention is effective in pyschotherapy, trauma healing, creative blocks, performance enhancement, creative enhancement, sports psychology and acting coaching.

Other features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims.

### BRIEF DESCRIPTION OF THE DRAWINGS

In the drawings, wherein similar reference characters denote similar elements through the several views:

FIG. 1 is a general flow diagram of brain mapping and processing of lesions in a recipient's brain in accordance with the subject invention; and

FIG. 2 is a detail flow diagram of brain mapping and processing of lesions in a recipient's brain as a result of trauma or YIPS-styled presentation, in accordance with a preferred embodiment of the subject invention.

### DETAILED DESCRIPTION OF THE PREFERRED EMBODIMENTS

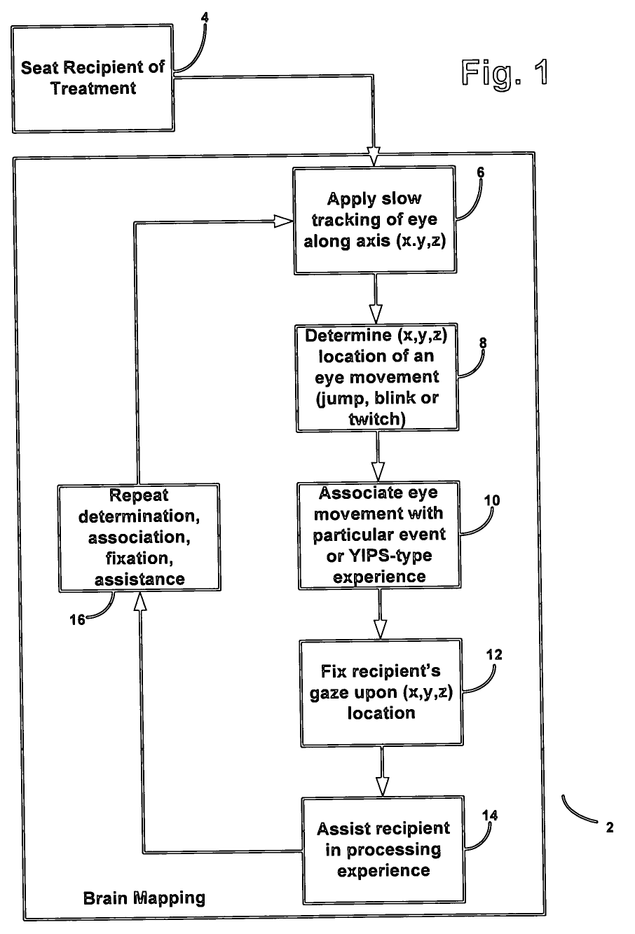

In accordance with the subject invention, FIG. 1 shows a flow diagram that represents the method and system of the subject invention by creating a brain map 2 of at least one and preferably various locations in the human brain in accordance with visual presentation by eye movement along the x and y axis, and preferably the z axis at locations that are indicative of lesions in the brain, for presentation to and processing by a recipient, in accordance with a preferred embodiment of the subject invention.

In particular, in FIG. 1, the method and system commences with a recipient seated for treatment at step 4. In this regard, the recipient is generally seated in a chair such that the recipient can support his/her head either by an extended hand and arm, or by slightly reclining such that the recipient can observe the practitioner and the stylus (either a hand, object, extended pointer with a tip, light emitting armature and the like) as the stylus is maneuvered through the relative axes as part of the process. Upon seating at step 4, the practitioner at step 6 applies slow tracking of the patient's eye motion along the x and y axes, and preferably as well the z axes, while having the recipient focus upon the tip of the stylus.

Once tracking proceeds at step 6, the practitioner observes the eye motion of the recipient in order to determine the location of eye movement at step 8. Typically a smooth eye motion is interrupted by a bodily reflex, typically an eye movement (either a jump, blink or twitch) and also including quick breaths, swallows, and facial and bodily twitches, at step 8, during the motion of the stylus in the x, y, and z axes at step 8. Such eye movements are indicative of a lesion in the brain, reflected by the eye movement. Lesions, as used herein, may be physically observable by diagnostic techniques, but generally refer to areas in the brain where brain functioning has been diminished or has ceased in processing one or more events. Such cessation of processing, recognized in the field, is, in accordance with the invention, the subject of eye movement. The instant invention predicates upon the association between such unprocessed events demonstrative of lesions and a bodily reflex, typically an eye movement (either a jump, blink or twitch) and also including quick breaths, swallows, and facial and bodily twitches, at various points in the visible eye movement range, and upon determination of the locations and provocation for discussion and analysis utilizing the techniques taught herein results in diminished effects of the trauma or other physiological or psychological causes and eventual elimination of the same by forced fixation of the recipient upon determined locations of eye movement together with verbalization of the underlying core event. Indeed, the recipient, upon fixation at a location in the eye spectrum, together with raising the felt sense of the underlying event and its processing, receives a heretofore unknown release of the event as it transits from conscious (often painful) discovery through visualization in color, black and white, and eventual elimination. Upon elimination, the brain passes through the stoppage or cessation of activity associated with the lesions, and returns to relative normalcy.

As shown in FIG. 1, the method and system involves determination of brain mapping on a point by point basis. Thus, once a location of an eye movement is determined in step 8, the movement is associated with a particular event or YIPS-type experience of the recipient by questioning at step 10 by the practitioner while the recipient's gaze is fixated on the location at step 12. In this manner, the recipient is assisted in processing the underlying experience relative to the fixated location of the eye(s) via step 14. As each location is mapped, the method and system of the instant invention is repeated for each subsequent location via step 16, by which the process is determined, association occurs, fixation on each subsequent location is provided, and assistance by the practitioner in processing is provided, via step 16.

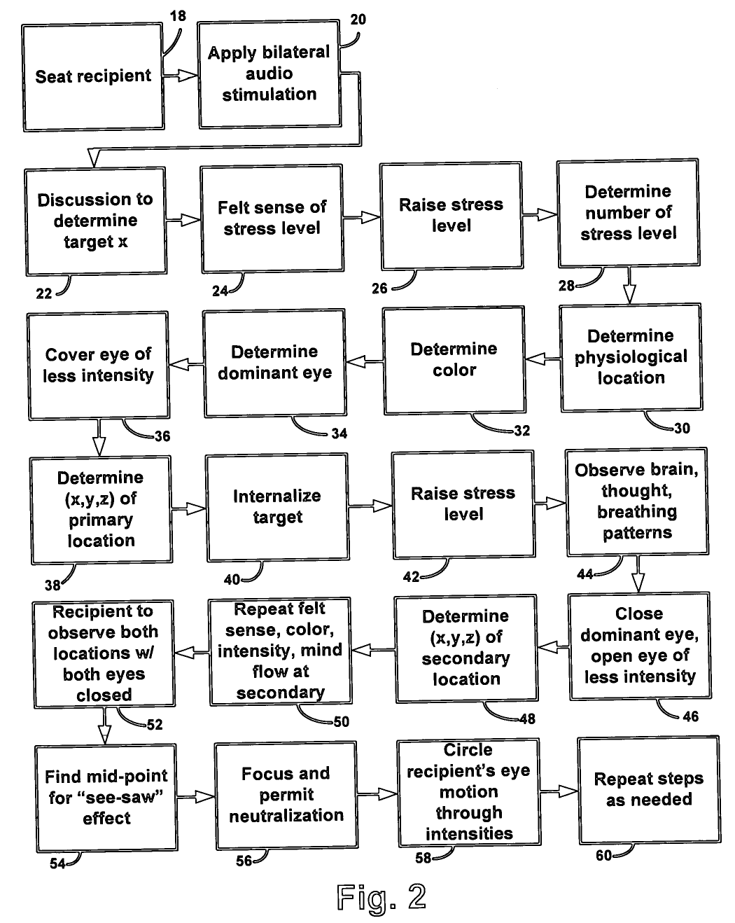

FIG. 2 shows a more detailed presentation of the method and system of the subject invention in accordance with a preferred embodiment of the subject invention. In particular, the recipient is seated at step 18, and, in this instance, bilateral audio stimulation is provided via step 20 for the duration of the experience. It has been discovered by the inventor herein, that bilateral auditory stimulation, provided by sound or music that is phase shifted between the left and right ears typically by recording sound or music in a manner in which each of the left side in succession is volumetrically raised and lowered such a sweeping sensation between the left and right sides of the recipient are experienced, stimulates mental processing of events that are theretofore representative of mental blockage by theretofore unprocessed events (e.g., traumatic experiences from those of early childhood to others like accidents, witnessing of non-normal events in military or terrorism (9/11), and the like). Bilateral auditory stimulation thus plays a pivotal role in overcoming trauma and YIPS-resultant events, and hence is applied herein in connection with the brain mapping method and system.

After application of bilateral audio stimulation at step 20, the recipient and practitioner discuss events to determine a target event via step 22. Thereupon, the recipient develops a felt sense of the stress level via step 24, and raises the stress level to a maximum at step 26. (It should be appreciated that raising the stress level at step 26 is contra-indicated by those practitioner of EMDR, who sweep through stressor events and typically do not compel the recipient to focus upon them, instead seeking a less intrusive method of operation, and hence teaching away from the techniques employed herein.)

Once the stress level is raised via step 26, the recipient is asked to determined the number of the stress level via step 28, typically on a scale of 1-10. The goal is to establish a high stress level above a baseline level, in order to permit efficacy of the method and system of the instant invention. As discussed in further detail hereinbelow, the higher the stress level, the greater the efficacy as the stress level is shown to be reduced to the point of ultimate elimination as the technique employed permits the recipient to process the underlying event that resulted in the lesion presented by the eye movement (jumping, blinking or twitching) and concomitant effects.

Upon determination of the stress level number at step 28, the recipient then determines the physiological location of “pain” which generally results from stimulation of the location. In this regard, a patient will likely have a felt sense of physical pain, at times in or about the eye(s) and in various location of the body, from head, to stomach or gut level, to chest, to back, and at times in the limbs from upper to lower body. A determination of the physiological location at step 30 is also a solid baseline as the method and system employed demonstrates a receding of that “pain” to the point of elimination resulting from the processing of the underlying traumatic event theretofore unprocessed.

Often the target also produces a felt sense of a color in the mind of the recipient, and that color is determined at step 32. It should be appreciated that human experience, especially traumatic events, are often translated by the human mind into a color, and that the method and system of the subject invention often reduces the felt sense of that color from bright, to dull, to black and white to eventual elimination. As the image (sensory, auditory, odor-based, or hallucinatory) underlying the traumatic event transits from “real” and in motion, to still to elimination, so, too, the color recedes as the brain becomes able to process the event.

In this regard, the subject invention adduces a dominant and secondary eye in relation to the lesionary influence of the underlying event. Thus, a dominant eye is determined at step 34 by requesting that the recipient close one eye and then the other to determine which one has a clearer image of the targeted underlying event, shown by a higher stress level from one to the other. Thus, the eye that results in the recipient's felt sense of a higher level of anxiety or “pain” associated with the trigger is determined to be the dominant eye via step 34. The eye of less intensity, i.e., the eye that is non-dominant or “secondary” is covered by the recipient of the method and system via step 36. Typically the eye is covered by the recipient's hand.

Thereafter, a determination of the primary location is adduced at step 38, utilizing substantially the same technique explained in connection with FIG. 1. Once the lcoation is determined, the recipient is requested to internalize the target at step 40, i.e., to focus upon the underlying event, essentially reliving it, as the recipient is compelled to focus on an event typically avoided by the recipient and hence heretofore the cause of a lesion or blockage of activity in the brain. Once the target is internalized at step 40, the recipient raises the stress level at step 42 to the maximum that the recipient can raise the same, and the practitioner observes the brain, though, and breathing patterns of the recipient at step 44. (Obviously, if the recipient commences dangerously aberrant behavior at this point, the process is ceased or reduced to avoid physiological damage to the recipient.)

Once the recipient is able to process the event through the dominant eye, the recipient next transfers to the secondary eye or eye of less intensity at step 46 by closing the dominant eye and opening the secondary eye. In this regard, the subject invention is predicated upon a discovery that the bilateral nature of the brain (in the absence of surgical intervention) provides pathways that transfer information from side to side and from side to back of the brain. Thus, the method and system of the subject invention also enables passage from side to side by moving from the dominant to secondary eye (and to the back, as demonstrated in connection with steps 54-58, discussed below). Once the secondary eye is opened and the primary closed at step step 46, a secondary location (x, y, z) is determined at step 48 utilizing the stylus in conformity with the steps heretofore outlined. Once the seocndary lcoation is determined at step 48, the recipient repeats the felt sense, color, intensity and mind flow of the target at the secondary location in the same manner as the primary location.

After completion of the secondary location, the recipient then closes both eyes and observes both locations virtually simultaneously with both eyes closed at step 52. It is at this point that both sides of the brain are “firing” virtually simultaneously (or in rapid succession) as the targeted event is now processed by both sides of the brain. A mid-point is determined (again by felt sense) between the primary and secondary location for a “see-saw” effect at step 54 wherein the recipient rocks between one side and the other as part of a processing stimulated by the method and system of the instant invention.

After the “see-saw” effect of step 54, the recipient focuses upon the event without shifting from one side to the other to permit neutralization at step 56. During the entire sequence, the numerical level of anxiety or “pain” is determined and generally reduces dramatically as the underlying trigger is processed by the recipient.

Lastly, with both eyes open and with the recipient focused upon the stylus, the stylus passes in a circle (clockwise or counterclockwise as determined for greatest efficacy) through intensities at step 58. Observably, as the stylus passes from dominant location to secondary location, the intensities are observed, and after a number of cycles, the anxiety or “pain” level is observed to decrease to the point of reaching a level below threshold. The steps are repeated via step 60 for each point in the map of the brain (dependent upon each trigger identified by the recipient with provocation by the practitioner) until the recipient is free of trigger events. To be sure, the recipient generally observes the trigger to pass from extreme acuity in visualization (full color and sharp edges), to lesser acuity and to eventual obscurity.

While there have been shown, described and pointed out fundamental novel features of the invention as applied to preferred embodiments thereof, it will be understood that various omissions and substitutions and changes in the form and details of the device illustrated and in its operation may be made by those skilled in the art without departing from the spirit of the invention. It is the intention, therefore, to be limited only as indicated by the scope of the claims appended hereto.

I claim:

1. A non-invasive method for permitting the processing of a triggering event by a recipient, comprising:
(a) applying a tracking of at least one eye along at least two axes selected from the group consisting of the x, y and z axes;
(b) determining a targeted location reflected by bodily reflex, typically an eye movement (either a jump, blink or twitch) and also including quick breaths, swallows, and facial and bodily twitches while tracking;
(c) associating said targeted location with a particular event in the recipient's life experience;
(d) fixing the recipient's gaze in said at least one eye upon the targeted location; and
(e) assisting the recipient in processing said particular event while maintaining the fixation of step (d).

2. The method of claim 1, wherein steps (a) through (e) are repeated to determine a brain map of all targeted locations and associated events.

3. The method of claim 1, wherein said particular event is selected from the group consisting of traumas, mental illness, YIP's-type occurrences, performance blocks, and creative blocks.

4. The method of claim 1, wherein said tracking is provided by the use of a stylus.

5. The method of claim 4, wherein said stylus is selected from the group consisting of a human hand, human finger, human fingers, a pointer, an telescopic extender device, a light emitter, an LED, a spectral diode, and a laser emitter.

6. The method of claim 1, further comprising seating the recipient prior to administering step (a).

7. The method of claim 1, further comprising the addition of application of bilateral auditory stimulation.

8. The method of claim 7, wherein said bilateral auditory stimulation comprises auditory sequences including sound and music recorded with phase shifting between the left and right channels.

9. The method of claim 8, wherein the phase shifting is subjectively determined.

10. The method of claim 9, wherein the phase shifting is objectively determined.

11. A treatment system for the detection, amelioration and treatment of psychological trauma and other mental disorders, and for performance and creative enhancement, of a recipient who has a mind, dominant eye and a secondary eye, comprising in relative sequence:
(a) a speaker system for application of bilateral auditory stimulation to the recipient;
(b) discussion with the recipient to determine a target for the system;
(c) felt sense determination of a stress level of the target;
(d) raising of said stress level and determining a number in association therewith;
(e) determining whether there exists a physiological location in association with said target;
(f) determining whether there exists a color in association with said target;
(g) determining the recipient's eye that is dominant and covering the recipient's secondary eye;
(h) a stylus for focusing the recipient's eye(s);
(i) employing said stylus to determine a primary location in the field of vision of the dominant eye that is associated with the target;
(j) having the recipient internalize the target and its felt sense, and raising the stress level experienced by the recipient while internalizing the felt sense of the target;
(k) observing the recipient's brain, thought and breathing patterns during step (j);
(l) closing recipient's dominant eye, and opening recipient's secondary eye;
(m) employing said stylus to determine a secondary location in the field of vision of the recipient's secondary eye that is also associated with the target;
(n) repeating felt sense, color, intensity while having the recipient focus upon the secondary location;
(O) having the recipient observe both the primary and secondary locations virtually simultaneously with both eyes closed;
(p) having the recipient determine a mid-point in the patient's mind for a “see-saw” effect;
(r) focusing the recipient to permit neutralization of the trigger;
(s) employing the stylus to have the patient's focus of both eyes travel in a relative circular pattern through each of the primary and secondary locations to complete neutralization while observing the increase and decrease in the stress levels; and
(t) repeating steps (b) through (g) and (I) through (s), as needed to ameliorate the trigger.

12. The treatment system of claim 11, wherein the speaker system comprises headphones placed about the ears of the recipient.
